(19) United States
(12) Patent Application Publication
(10) Pub. No.: US 2007/0233131 A1
(43) Pub. Date: Oct. 4, 2007

(54) Apparatus and method of creating an intervertebral cavity with a vibrating cutter

(75) Inventors: John K. Song, Chicago, IL (US); Jeffrey Gordon, Saratoga Springs, NY (US)

Correspondence Address: John K. Song, #4508, 474 North Lakeshore Drive, Chicago, IL, 60611, US

(73) Assignee: Vermillion Technologies, LLC, Chicago, IL (US)

(21) Appl. No.: 11/711,562

(22) Filed: Feb. 27, 2007

Related U.S. Application Data
(60) Provisional application No. 60/777,261, filed on Feb. 28, 2006.

(51) Int. Cl. A61B 17/00

(52) U.S. Cl. 606/79

(57) ABSTRACT

An apparatus and method for creating a space of defined length, height, width and shape with a vibrating cutting apparatus in preparation for receiving an interbody spinal implant or graft of known size and configuration is disclosed.

### CROSS-REFERENCE TO RELATED PATENT APPLICATION

This application claims priority to and the benefit of, pursuant to 35 U.S.C. §119(e), U.S. provisional patent application Ser. No. 60/777,261, filed Feb. 28, 2006, entitled “Apparatus and method of creating an intervertebral cavity with a vibrating cutter” by John K Song and Jeffrey David Gordon and is incorporated herein by reference in its entirety.

### FIELD OF THE INVENTION

The present invention generally relates to a vibratory or orbital device for creating a cavity between or within bones of the human body.

### BACKGROUND OF THE INVENTION

Surgery of the bones of the human body has been greatly facilitated with the advent of powered tools, most notably surgical drills. Most commonly, traditional rotating surgical drills are similar to their non-surgical counterparts and incorporate a motor rotating a drive shaft about its long axis and a cutting tool attached to the drive shaft. Occasionally the drive shaft is powered by pneumatics or other power source. The cutting tool rotates around the long axis of its shaft in a continuous motion and when applied to bone will remove material.

More recently, ultrasonic surgical drills have been introduced. These typically incorporate piezoelectric crystals to actuate a drive shaft in a linear manner along its long axis. Ultrasound may comprise a defined frequency range but for the purposes of the present invention we define vibrational and orbital motions to include ultrasonic, subsonic and suprasonic ranges. Ultrasonic drills abrade bone surfaces in a controlled manner with none of the problems of traditional rotating surgical drills such as “skipping” (whereby the cutting tool grabs the bone surface and “skips” away) or wrapping up soft tissue.

In addition, ultrasound surgical drills may employ cutting tools which are asymmetric about any axis, especially about the long axis of the drive shaft and thus more advantageous geometries may be used for specific surgical procedures.

For the purposes of the present invention, drills which rotate continuously in one direction along the long axis of the drive shaft are referred to as “rotating” and drills which move the cutting tool in a substantially linear or rotational back-and-forth manner along any axis (FIG. 14) are referred to as “vibratory” or “ultrasonic”. Alternatively, drills or similar tools which cause the cutting tool to move in a non-linear, continuous, planar path (FIG. 14, XY, XZ, YZ planes) are referred to as “orbital”. An example of orbital motion is an orbital sander for finishing wood surfaces.

For the purposes of the present invention, “bones” refers to any two or more anatomically distinct bones or two or more pieces of the same bone. It is not intended by specific mention of any particular bone within this work to limit the scope of the present invention.

### SUMMARY OF THE INVENTION

The present invention is a vibrating cutter for surgically creating a cavity between, or partially within, two bones. Specifically, the invention creates the cavity by means of a vibratory actuator such as an ultrasound actuator. By eliminating standard rotating cutting means such as mills or drills, the invention offers improved safety and is capable of creating a geometrically complex cavity with a relatively simple cutting tool. The preferred embodiment of the invention has utility in creating a precise cavity in the intervertebral space between two vertebrae of the spine.

It is an object of the present invention to provide an instrument for surgically creating a cavity between, or partially within, two bones.

It is another object of the present invention to provide an instrument for facilitating implantation of an interbody device, such as a fusion device, a total disc arthroplasty, a bone graft, or a nucleus replacement, or for facilitating implantation of a facet replacement.

It is another object of the present invention to provide an instrument incorporating a sizer/introducer which (1) acts to distract the bones, (2) acts to aid in selection of the cutter and implant or bone graft size, and (3) acts as a guide for safe, precise placement and orientation of the cutting tool.

These and other objects of the present invention will become apparent from a review of the accompanying drawings and the detailed description of the preferred embodiments.

### BRIEF DESCRIPTION OF THE DRAWINGS

FIG. 1 is a perspective view of the vibrating cutter and vibrational actuator assembly of the present invention.

FIG. 2a is a perspective view of the vibrating cutter illustrating insertion into a disc space.

FIG. 2b is a perspective view of the vibrating cutter showing the cutter inserted into a disc space.

FIG. 2a is a perspective view of the vibrating cutter illustrating withdrawal from a disc space and the cut created in the vertebral endplates.

FIG. 3 is a perspective view of the vibrating cutter of the present invention.

FIG. 4 is a perspective detailed view of the vibrating cutter of the present invention.

FIG. 5 is an alternate embodiment of the vibrating cutter.

FIG. 6 is an alternate embodiment of the vibrating cutter with circular shape and pyramidal cutting teeth

FIG. 7 is an alternate embodiment of the vibrating cutter with a protrusion incorporated on the top and bottom surfaces.

FIG. 8 is an alternate embodiment of the vibrating cutter with a cylindrical shape.

FIG. 9 is an alternate embodiment of the vibrating cutter with an angular shape and conical cutting teeth.

FIG. 10 is an alternate embodiment of the vibrating cutter with a partial conical shape and holes through the cutter.

FIG. 11 is an alternate embodiment of the vibrating cutter with a keel incorporated on the top and bottom surfaces and teeth on a portion of the side surfaces

FIG. 12 is an alternate embodiment of the vibrating cutter with a hollow cutting body, holes through the cutting surfaces and cutting teeth on the front surfaces.

FIG. 13 is the reverse view of the embodiment shown in FIG. 13.

FIG. 14 is a perspective view of the vibrating cutter showing some of the possible axes of translations and rotations of vibration.

FIG. 15 is a perspective view of an alternate embodiment of the vibrating cutter with a spacer and an enclosed, rotatable cutter for cutting a protrusion in the vertebral endplates.

FIG. 16 is a perspective view of an alternate embodiment of the vibrating cutter with a spacer and an enclosed, partially rotatable cutter for cutting a protrusion in the vertebral endplates.

FIG. 17 is a sectioed front view of the alternate embodiment of the vibrating cutter shown in FIG. 16.

FIG. 18a is a perspective view of the sizer/introducer.

FIG. 18b is a perspective view of the sizer/introducer and vibrational actuator-vibrating cutter assembly illustrating the guiding action of the sizer/introducer.

FIG. 18c is a perspective view of the sizer/introducer and vibrational actuator-vibrating cutter assembly.

### DETAILED DESCRIPTION OF THE PREFERRED EMBODIMENTS

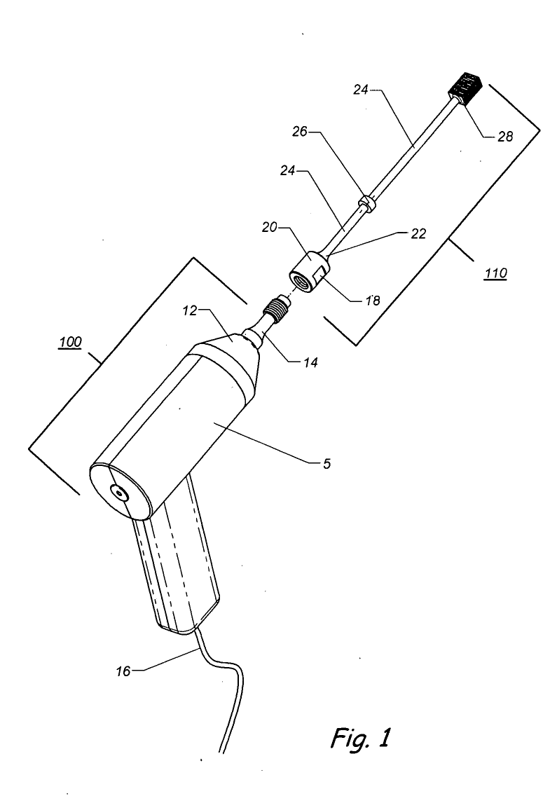

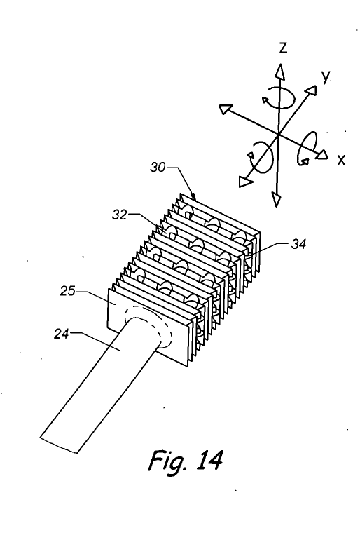

Referring to FIG. 1, a vibrational actuator 100 and vibrating cutter 110 assembly is shown. Vibrational actuator 100 is of the type demonstrated in the prior art, such as the “Ultrasonic Nailing and Drilling Apparatus” described by Hur (U.S. Pat. No. 6,204,592). The vibrational actuator 100 is capable of producing vibratory motion in at least one degree of freedom (DOF) as described in FIG. 14 below. The frequency of the vibratory motion is preferably in the ultrasonic range but alternatively can be sub-ultrasonic or super-ultrasonic. The actuator is preferably powered by a source of electricity such as an AC or DC power source or battery, but can alternatively be gas powered such as a pneumatic actuator. The actuator is preferably driven by a piezo-ceramic material such as PZT, or a magnetostrictive material such as Terfonol-D. The preferred embodiment is a vibrational actuator 100 that is connected to an electrical energy source (not pictured) by an electric cord 16. An actuation tip 14 protruding from a body 5 or an end cap 12 of the vibrational actuator 100 is constructed to engage a driving end 20 of the vibrating cutter. The preferred connection means is mechanical threads, but alternatively a mechanical quick-release mechanism, a chuck, a collet, a pin, a fastener, multiple fasteners, a clamp or other mechanical connection means can be encorporated. In the preferred embodiment, internal threads on the driving end 20 of the vibrating cutter 110 are engaged with external threads on the actuation tip 19 by using a wrench (not pictured) engaged in flats 18 on driving end 20 of the vibrating cutter 110. Counter rotation during assembly is provided by means internal to the vibrational actuator 100 but can alternatively be provided by a feature incorporated into the construction of actuation tip 14. An elongated shaft 24 transmits the vibratory motion to a cutting tip of the vibrating cutter. Reduction in diameter, where necessary to facilitate insertion into the body cavity of the patient, may be strengthed by inclusion of a fillet 22, however a chamfer may be utilized. Strengthening means may not be necessary. A depth stop 26 is preferably incorporated into shaft 14 to control the depth of penetration of the vibrating cutter into the intervertebral space. Alternatively, depth can be controlled by other means including radiography.

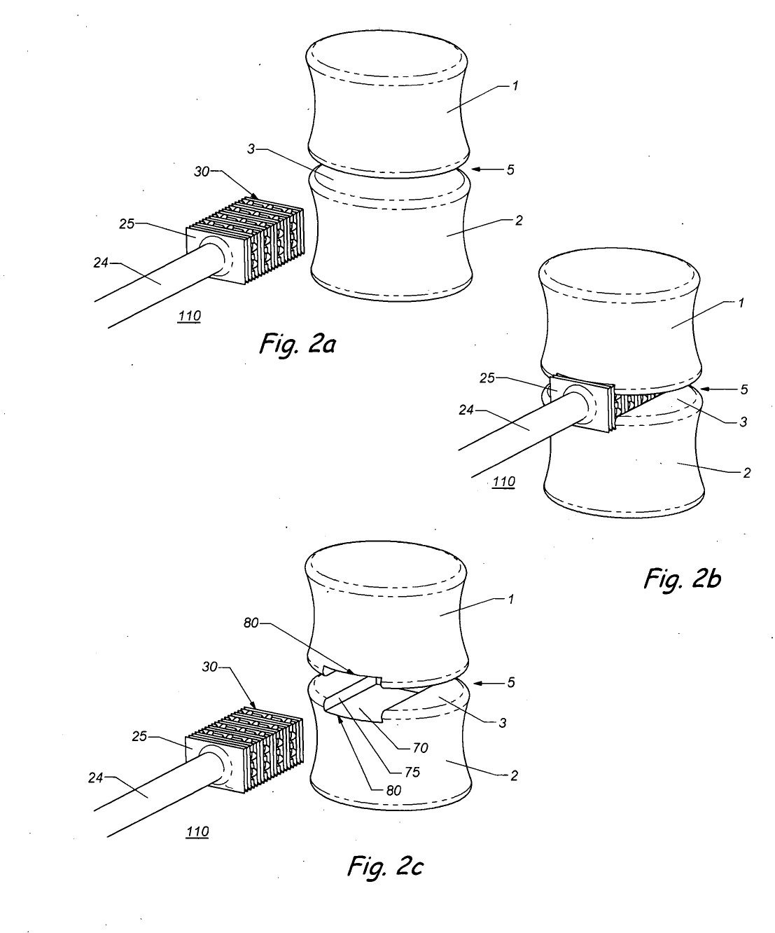

FIGS. 2a, 2b and 2c demonstrate the procedure of preparing an intervertebral space for an implant(s). FIG. 2a shows a superior vertebral body 1, an inferior vertebral body 2, a disc space 5 and vibrating cutter 110. Shaft 24 is aligned such that cutting teeth 30 will cut the appropriate contour into endplate(s) 3 of the disc space 5. FIG. 2b shows the vibrating cutter 110 advanced into disc space 5. FIG. 2c shows the cut made in superior vertebral body 1 and inferior vertebral body 2 and vibrating cutter 110 withdrawn from disc space 5. The resulting cut forms a cavity 70 in superior vertebral body 1, and inferior vertebral body 2 which has side walls 75 and a front edge 80 which can be of various shapes as will be demonstrated in the following figures.

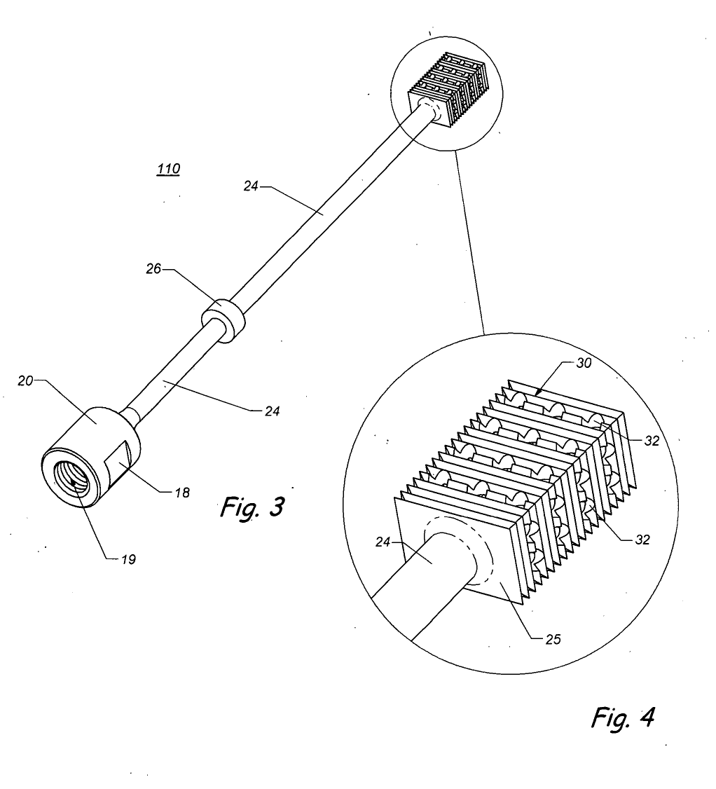

FIGS. 3 and 4 show a preferred embodiment of vibrating cutter 110 with driving end 20 as described above, shaft 24, and depth stop 26 to engage into an adjustable or non-adjustable stop incorporated into a guide mechanism (not pictured). In this embodiment, cutting teeth 30 exist on all sides of the cutting head except a trailing surface 25. Holes 32 are incorporated into the cutting head to collect chips of bone and to facilitate the cutting process.

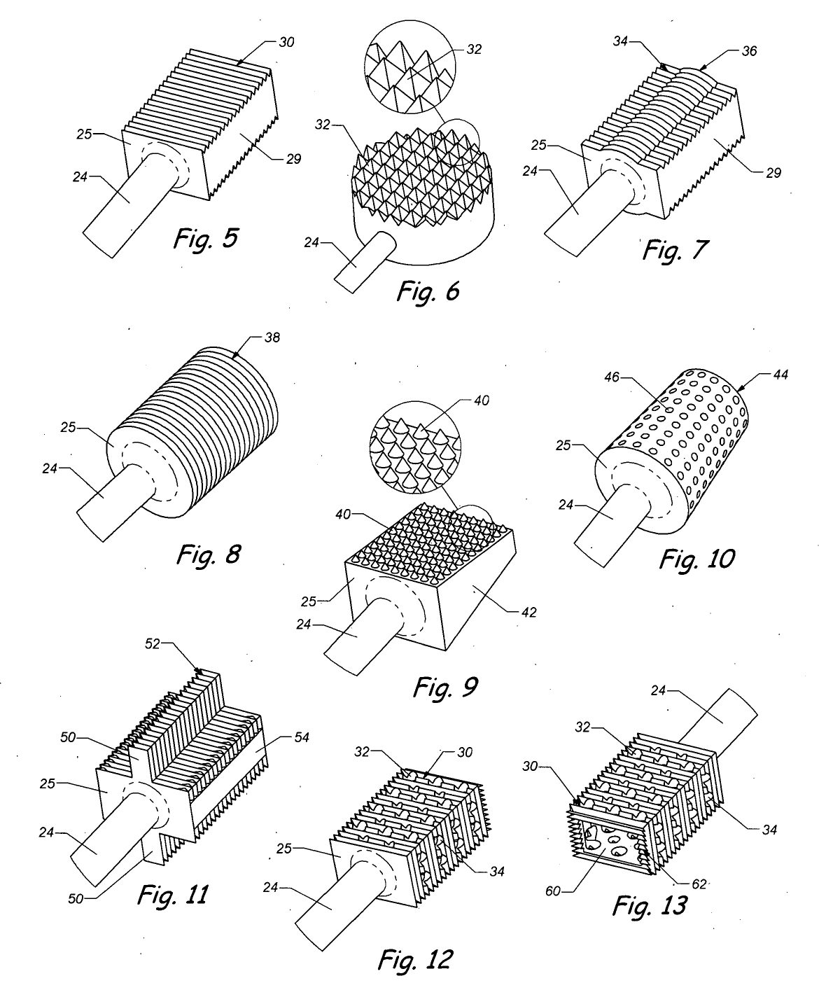

FIG. 5-13 are alternative embodiments of the cutting head of vibrating cutter 110. All embodiments of vibrating cutter 110 could either be inserted while cutting, or alternatively could be inserted into disc space 5 while not activated by vibratory actuator 100 and then activated once in disc space 5 so that the anterior surface of superior vertebra 1 and inferior vertebra 2 would not be cut and only endplates 3 would be cut. Many features are demonstrated, but a combination of any of these features into a single cutting head can be utilized. Configurations are for demonstration only and are not intended to limit the scope of the cutting head geometry. FIG. 5 shows a cutting head of rectangular cross-section with cutting teeth 30 on top and bottom surfaces. Side surfaces 29 do not incorporate cutting teeth. FIG. 6 shows a cutting head with a circular shape with pyramidal teeth 32 on the top surface only. A cutting head with teeth on one surface only would permit cutting one endplate at a time and may require reinsertion after a 180 degree rotation of vibrating cutter 110 to cut an opposing endplate. FIG. 7 shows a cutting head with a protrusion incorporated into top and bottom surfaces to create a space for a corresponding protrusion incorporated into an implant. Cutting teeth 34 are shown on top and bottom surfaces but not front surface 36 or trailing surface 25. FIG. 8 shows a cutting head with a substantially cylindrical shape to create a space for a correspondingly shaped implant such as a fusion cage. Cutting teeth 38 would be incorporated, but may or may not continue all the way to trailing surface 25. FIG. 9 shows a substantially wedge shaped cutting head to create a space for a correspondingly shaped implant which creates a lordosis or kyphosis angle in disc space 5. Conical teeth 40 are incorporated in top and bottom surfaces, but may also be incorporated into side surface 42 and/or the front surface. FIG. 10 shows a cutting head with a partial conical shape to create a space for a correspondingly shaped implant which creates a lordosis or kyphosis angle in the corresponding disc space 5. Holes 46 are incorporated into the cutting surfaces to aid in cutting endplate bone material. FIG. 11 shows a cutting head with a keel type protrusions 50 to create a space for a correspondingly shaped implant such as a disc replacement with a keel. Teeth 52 are incorporated into top, bottom, and protrusion surfaces and a portion of side surfaces. Surfaces 54 are devoid of cutting teeth to aid in insertion. FIGS. 12 and 13 show a cutting head with a substantially hollow shape and holes 32 to aid in cutting and/or to aid in collection of bone material for grafting purposes. Interior surfaces 60 are meant to collect bone chips created in the cutting process. Front surface teeth 62 are incorporated in this embodiment and could also be incorporated in any of the preceding cutting head embodiments. FIG. 13 is a reverse angle view of FIG. 12 to show interior surfaces 60 and front surface teeth 62.

FIG. 14 shows a cutting head of vibrating cutter 110 and some of the possible motions of vibration possible. Vibrational actuator 100 will be capable of translating vibrating cutter 110 along, or rotating it about, axes X, Y or Z or any combination of these motions or orbital motion.

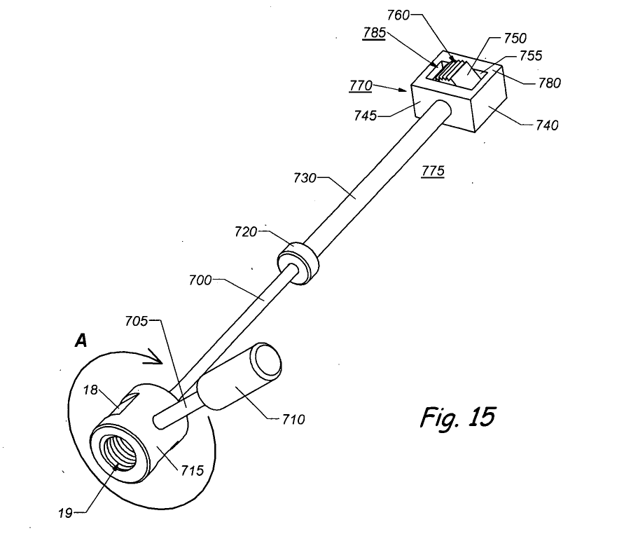

FIG. 15 shows vibrating cutter 110 with a moveable protrusion cutter. A spacer 775 incorporating side walls 740, a rear wall 745, and a substantially flat surface 780 can be inserted into disc space 5 with a protrusion cutting head 785 oriented so that substantially flat surface 750 is parallel to substantially flat template surface 780. Shaft 700 is rigidly connected to protrusion cutting head 785 and a driving end 715 and is constructed to be capable of rotating within hollow shaft 730 of template 775. After insertion, handle 710 can be rotated through any angle A so that cutting teeth 760 cut a recess into endplates 3 which substantially match a corresponding shape in an implant. Template 775 may further incorporate a depth stop 720 to limit the depth of the protrusion cut in superior vertebra 1 and inferior vertebra 2 with respect to their anterior surface.

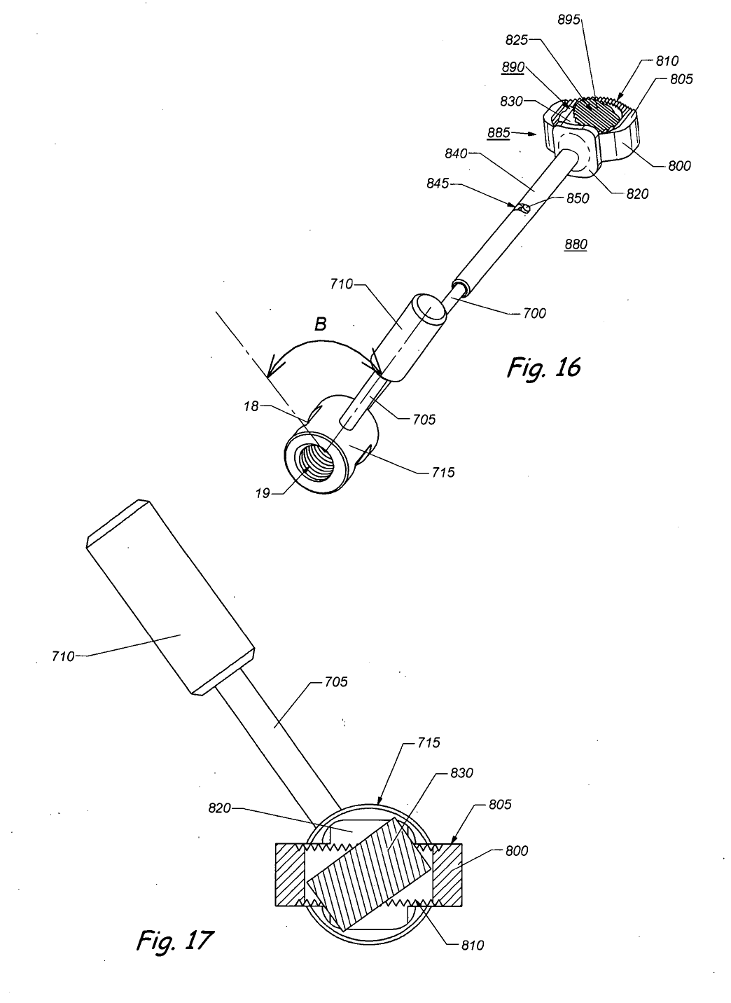

FIGS. 16 & 17 show another embodiment of vibrating cutter 110 with a moveable protrusion cutter similar to the embodiment shown in FIG. 15. A spacer 880 has an intervertebral portion 885 with side walls 800, cutting teeth 810, a substantially flat guide surface 805 and a depth stop 820, is attached to a hollow shaft 840 with a slot 845. A shaft 700 is rigidly connected to a protrusion cutting head 890 with a protrusion cutting surface 895 with cutting teeth 825, and is also rigidly connected to a driving end 715. A handle 710 is connected to driving end 715 by means of a shaft 705. Shaft 700 is able to rotate within hollow shaft 840 when handle 710 is rotated. Spacer 880 is inserted into disc space 5 until depth stop 820 contacts the anterior surface of either superior vertebra 1 or inferior vertebra 2 or both. Spacer 880 is inserted with a protrusion cutting surface 895 oriented so that protrusion cutting surface 895 is substantially parallel to guide surface 805. After insertion, handle 710 is rotated through an angle B so that cutting teeth 825 on protrusion cutting surface 895 cut a recess into endplates 3 which substantially matches a corresponding protrusion in an implant. A pin 850 moves within slot 845 which acts to limit the range of rotation of protrusion cutting head 890. FIG. 17 is a front view of the assembly shown in FIG. 16.

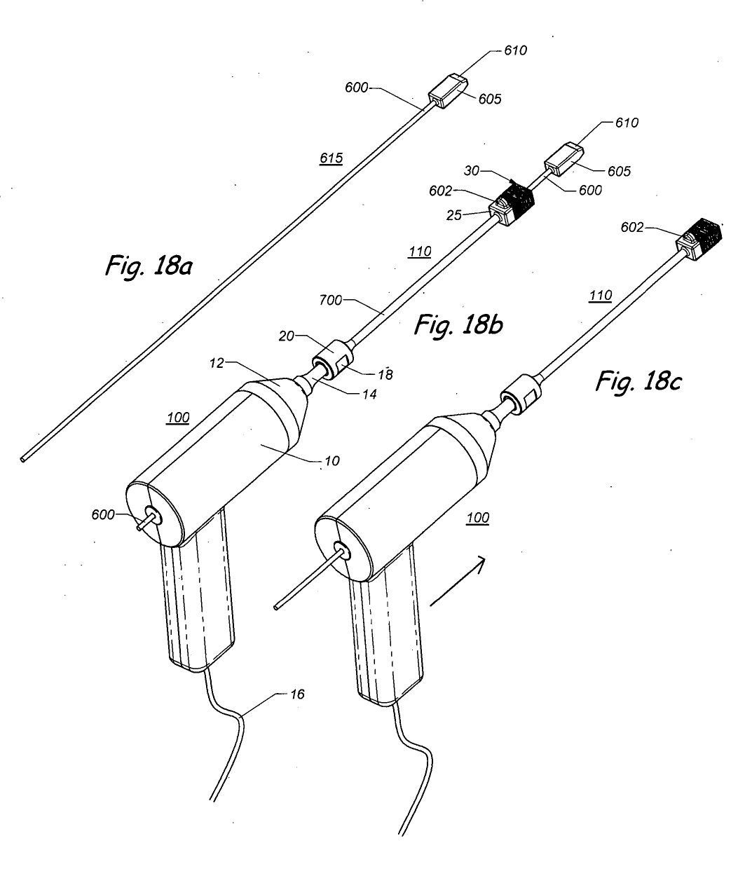

FIGS. 18a, 18b, and 18c show an alternative method of creating an intervertebral space. A sizer/introducer 615 with an elongated shaft 600 and an intervertebral head 605 with sloped front surface(s) 610 is first introduced into disc space 5. Front surface(s) 605 may be in the form of a “bullet” tip. Vibrating cutter 110 is cannulated so that it fits over shaft 600. In the figure, Vibrational actuator 100 is also cannulated, but this may not be necessary. Sizer/introducer 615 therefore functions as a guide to control the placement and orientation of the cut. The vibrational actuator 100 and vibrating cutter 110 assembly is slid over shaft 600 and cuts a space into superior vertebra 1 and/or inferior vertebra 2 as shown in FIGS. 18b and 18c. Vibrational cutter 110 may also incorporate a depth stop 602 to limit travel into disc space 3.

What is claimed is:

1. A device for creating a cavity between or at least partially within two or more bones or bone surfaces by means of a vibrating cutting tool where said cutting tool is shaped substantially like the desired bone cavity

2. The device of claim 1, where said cutting tool is placed adjacent to, and creates a cavity between, said bones

3. The device of claim 1, where said cutting tool is placed adjacent to, and creates a cavity within, said bones

4. The device of claim 1, where said cutting tool is passed along or within a cutting guide

5. The device of claim 4, where said cutting tool is rotated within or around said cutting guide to create said cavity

6. The device of claim 4, Where said cutting guide also acts as a spacer to maintain a fixed distance between said bones

7. The device of claim 1, where said cavity is shaped to fit an implant

8. The device of claim 7, where said implant is made of a biologically compatible material

9. The device of claim 4, where said cutting tool has a cavity for said cutting guide

10. The device of claim 1, where said cutting tool has a cavity for collection of bone debris

11. The device of claim 1, where said cutting tool has perforations through its bone-contacting surface for passage of irrigation or bone debris

12. The device of claim 1, further comprising a depth stop

13. A device for creating a cavity between or at least partially within two or more bones or bone surfaces by means of an orbiting cutting tool where said cutting tool is shaped substantially like the desired bone cavity

14. The device of claim 13, where said cutting tool is placed adjacent to and creates a cavity between said bones

15. The device of claim 13, where said cutting tool is placed adjacent to and creates a cavity within said bones

16. The device of claim 13, where said cutting tool is passed along or within a cutting guide

17. The device of claim 16, where said cutting tool is rotated within or around said cutting guide to create said cavity

18. The device of claim 16, where said cutting guide also acts as a spacer to maintain a fixed distance between said bones

19. The device of claim 13, where said cavity is shaped to fit an implant

20. The device of claim 19, where said implant is made of a biologically compatible material

21. The device of claim 16, where said cutting tool has a cavity for said cutting guide

22. The device of claim 13, where said cutting tool has a cavity for collection of bone debris

23. The device of claim 13, where said cutting tool has perforations through its bone-contacting surface for passage of irrigation or bone debris

24. The device of claim 13, further comprising a depth stop
